(19) United States
(12) Patent Application Publication
(10) Pub. No.: US 2003/0120270 A1
(43) Pub. Date: Jun. 26, 2003

(54) Ablation therapy

(75) Inventor: David E. Acker, Setauket, NY (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Transurgical, Inc., Setauket, NY

(21) Appl. No.: 10/131,755

(22) Filed: Apr. 23, 2002

Related U.S. Application Data
(60) Provisional application No. 60/285,845, filed on Apr. 23, 2001.

(51) Int. Cl. A61B 18/18

(52) U.S. Cl. 606/41

(57) ABSTRACT

Methods and apparatus for treating cardiac arrhythmias by ablating myocardial fibers within a pulmonary vein through use of a catheter. For example, the ablative element of the catheter is rotated around an axis to ablate a partial or complete loop of tissue within the pulmonary vein so as to block the transmission into the cardiac tissue of electrical signals originating or propagating from myocardial fibers within a pulmonary vein. In other examples, signals from the catheter are monitored to determine whether the ablative element is in contact with the wall of the pulmonary vein. Additional apparatus allow precise angular positioning of the ablative element within the lumen of the pulmonary vein. Apparatus and methods for detecting the properties of the tissue within the pulmonary vein, locating myocardial fibers, selectively ablating such fibers, and determining if such fibers are ablated are also disclosed.

### CROSS REFERENCE TO PRIOR APPLICATIONS

The present application claims the benefit of U.S. Application No. 60/285,845, filed Apr. 23, 2001, the disclosure of which is hereby incorporated by referenced herein.

### BACKGROUND OF THE INVENTION

The present invention relates to apparatus and methods for treatment of cardiac arrhythmias such as atrial fibrillation.

The normal contractions of the heart muscle arrive from electrical impulses generated at a focus within the heart and transmitted through the heart muscle tissue or “myocardial” tissue. In some individuals, fibers of myocardial tissue extend from the wall of the left atrium along the wall of the pulmonary vein. For example, the tissue of the pulmonary vein normally merges with the myocardial tissue of the heart wall at a border near the opening or ostium of the pulmonary vein. In some individuals, however, elongated strands of myocardial tissue extend within the wall of pulmonary vein in the distal direction (away from the heart) so that the strands of myocardial tissue project beyond the normal border. It has been recognized that atrial fibrillation can be caused by an abnormal electrical focus in such strands of myocardial tissue. Electrical signals propagate from such an abnormal focus proximally along one or more strands of myocardial tissue. Because these strains of myocardial tissue merge with myocardial tissue of the heart wall, the abnormal electrical signals propagate through the myocardial tissue in heart wall itself, resulting in abnormal contractions.

It has been recognized that this condition can be treated by locating the abnormal focus and ablating (i.e., killing or damaging) the tissue at the focus so that the tissue at the focus is replaced by electrically inert scar tissue. However, the focus normally can be found only by a process of mapping the electrophysiological potentials within the heart and in the myocardial fibers of the pulmonary vein. There are significant practical difficulties in mapping the electrical potentials. Moreover, the abnormal potentials which cause atrial fibrillation often are intermittent. Thus, the physician must attempt to map the abnormal potentials while the patient is experiencing an episode of atrial fibrillation.

Another approach that has been employed is to ablate the tissue of the heart wall, so as to form a continuous loop of electrically inert scar tissue extending entirely around the region of the heart wall which contains the ostium of the pulmonary veins, so that the abnormal electrical impulses do not propagate into the remainder of the atrial wall, outside the loop. In a variant of this approach, a similar loop like scar can be formed around the ostium of a single pulmonary vein or in the wall of the pulmonary vein itself proximal to the focus so as to block propagation of the abnormal electrical impulses. Such scar tissue can be created by forming a surgical incision; by applying energies such as radio frequency energy, electrical energy, heat, intense light such as laser light; cold; or ultrasonic energy. Chemical ablation agents also can be employed. Techniques which seek to form a loop-like lesion to form a complete conduction block between the focus and the major portion of the myocardial tissue are referred to herein as “loop blocking techniques.”

Loop blocking techniques are advantageous because they do not require electrophysiological mapping sufficient to locate the exact focus. However, if a complete loop is not formed, the procedure can fail. Moreover, ablating complete, closed loops without appreciable gaps presents certain difficulties. Thus, some attempts to form a complete loop of ablated tissue around the entire circumference of the pulmonary vein have left significant unablated regions and thus have not formed a complete conduction block. Other attempts have resulted in burning or scarring of adjacent tissues such as nerves. Moreover, attempts to form the required scar tissue using some types of ablation instruments such as radio frequency ablation and unfocused ultrasonic ablation have caused thromboses or stenosis of the pulmonary vein. The potential for these undesirable side effects varies directly with the amount of tissue ablated. Moreover, the amount of energy which must be applied in an ablation procedure varies directly with the amount of tissue ablated. Particularly where an ablation element must be introduced into the heart through a catheter, the size of the ablation element and hence the energy delivery capacity per unit time of the ablation element is limited. While these difficulties can be alleviated or eliminated by the use of focused ultrasonic ablation as taught, for example, in copending, commonly assigned U.S. Provisional Patent Application No. 60/218,641 filed Jul. 13, 2000, now U.S. patent application Ser. No. 09/905,227 “Thermal Treatment Methods and Apparatus With Focused Energy Application”; Ser. No. 09/904,963 “Energy Application With Inflatable Annular Lens”; and Ser. No. 09/904,620 “Ultrasonic Transducers,” the disclosure of which are incorporated by reference herein, further alternatives would be desirable.

### SUMMARY OF THE INVENTION

One aspect of the present invention provides apparatus for treating tissue adjacent a tubular anatomical structure having a lengthwise direction as, for example, for treating tissue of the pulmonary vein wall or tissue of the heart wall in the region surrounding the ostium of the pulmonary vein. The apparatus according to this aspect of the present invention preferably includes a carrier catheter and an anchor. When the device is in an operative condition, the carrier catheter is linked to the anchor so that the carrier catheter is movable with respect to the anchor over a predetermined path of motion. Preferably, the carrier catheter is rotatable with respect to the anchor around a first axis. The carrier catheter may be substantially constrained against movement relative to the anchor transverse to the first axis. The anchor is adapted to engage the wall of the tubular anatomical structure, or another adjacent bodily structure, so that the first axis extends generally in the lengthwise direction of the tubular anatomical structure. The apparatus also includes a local treatment device adapted to confront tissue of the subject at a point and treat tissue at one or more spots adjacent such point. When the device is in an operative condition, the local treatment device is remote from the first axis. The local treatment device desirably projects from the carrier catheter in a direction transverse to the first axis. Thus, the treatment device will trace a generally arcuate path around the first axis when the carrier catheter is rotated relative to the anchor. The local treatment device may include an ultrasonic emitter, RF ablation electrode, optical fiber, chemical applicator or even a mechanical device such as a blade adapted to engage tissue to a controlled depth.

In a particularly preferred arrangement, the anchor is affixed to an elongated guide structure such as a guide wire. The carrier catheter desirably has a first lumen which receives the guide wire so that the carrier catheter is rotatable about the guide wire. In one arrangement, the local treatment device is carried on a treatment catheter separate from the carrier catheter. The carrier catheter may have a separate carrier catheter lumen extending generally parallel to the guide lumen. A port may be provided in the side wall of the carrier catheter adjacent the distal end thereof. The port communicates with the treatment catheter lumen. In use, the treatment catheter is forced distally within the treatment catheter lumen after the carrier catheter is in place. As the treatment catheter is forced distally, the distal end of the treatment catheter bends outwardly through the hole in the carrier catheter. The local treatment device is carried at or near the distal end of the treatment catheter so that the local treatment device is moved radially outwardly, away from the guide lumen when the treatment catheter is forced distally. In other arrangements, the local treatment device may be carried on a flexible member mounted to the carrier catheter itself and the flexible member may be deformed so as to bend it outwardly, away from the guide lumen.

The treatment catheter or member carrying the local treatment device desirably is provided with a sensor such an electrode which can be used to detect engagement of the treatment catheter or other member with the tissue. For example, when such an electrode is brought into engagement with cardiac tissue, the electrode will pick up electrophysiological potentials present in the cardiac tissue.

Most preferably, the apparatus includes a device for controlling or monitoring the rotation of the carrier catheter relative to the anchor or relative to the patient himself. For example, a device for converting linear motion to rotary motion may be connected between the carrier catheter and the guide structure. One such device, commonly referred to as a “Yankee screwdriver” or “New England screwdriver” mechanism includes a generally helical cam surface on one member and a cam follower on the other member so that as the guide catheter is moved distally and proximally along the guide structure, the guide catheter rotates by a known amount per unit movement. In another arrangement, the carrier catheter or treatment catheter is provided with a sensor arranged to detect a magnetic or electromagnetic field and to provide one or more signals which vary depending upon the alignment of the sensor with the field. Provided that a constant field or field varying in known manner is imposed through the patient, the rotation of the carrier catheter can be monitored by monitoring the one or more signals from such a sensor.

In a particularly preferred arrangement, the apparatus includes a sensor for determining properties of tissue surrounding the tubular anatomical structure. The sensor desirably is linked to the carrier catheter when the sensor is in an operative condition. The sensor may be, for example, an ultrasonic, electrical, optical or other device. Thus, by rotating the first axis while the sensor is operating, the tissue surrounding the tubular anatomical structure can be mapped. In particular, for apparatus intended to be used in treatment of atrial fibrillation, the sensor may be operative to detect differences between regions of a pulmonary vein wall which contain myocardial fibers and other regions which do not contain myocardial fibers. As described in co-pending, commonly assigned U.S. provisional patent application Ser. No. 60/265,480, filed Jan. 31, 2001, now U.S. patent application Ser. No. 10/062,693 “Pulmonary Vein Ablation With Myocardial Tissue Locating,” the disclosure of which is hereby incorporated by reference herein, the myocardial fibers typically are located in only a portion of the pulmonary vein wall. Once the fibers are located, the treatment device can be actuated to ablate or otherwise treat the vein wall only over a portion of the vein wall circumference. The sensor may be a local sensor arranged to detect a property of the tissue in a local region immediately adjacent the sensor. Thus, by actuating the sensor while rotating the carrier catheter, a map of tissue property against rotational position of the carrier catheter can be acquired by plotting the signals acquired from the sensor against rotational position of the carrier catheter. The sensor may be carried on the treatment catheter. Indeed, the elements discussed above with reference to the treatment catheter may also serve as the sensor. For example, where an electrode is provided on the treatment catheter, the electrode can be used to map electrical potentials around the circumference of a pulmonary vein. Alternatively or additionally, the same ultrasonic transducer used in an ultrasonic ablation device can be used as a ultrasonic mapping element.

Further aspects of the present invention provide methods of treating tissue adjacent a tubular anatomical structure as, for example, the tissue of a pulmonary vein wall or the tissue of the heart surrounding the ostium of the pulmonary vein. Methods according to this aspect of the present invention desirably include the steps of positioning an anchor within the tubular anatomical structure and moving the carrier catheter along a predetermined path of motion relative to the anchor, as, for example, by rotating the carrier catheter with respect to the anchor around a first axis extending generally in the lengthwise direction of the anatomical structure, so that a local treatment device takes a predetermined path along the tissue. For example, a local treatment device projecting from the carrier catheter in a direction transverse to the first axis traces a generally arcuate path centered on the first axis over the tissue surrounding the anatomical structure, and actuating the local treatment device. Methods according to this aspect of the invention may include further steps of monitoring or controlling the position of the carrier catheter relative to the anatomical structure, as by monitoring or controlling the position of the carrier catheter relative to the anchor, such as the rotational position of the carrier catheter, and may also include mapping properties of the tissue along the path as, for example, by using a local sensor linked to the carrier catheter as discussed above in connection with the apparatus.

### BRIEF DESCRIPTION OF THE DRAWINGS

FIG. 1 is a cut-away view of the ostium and a portion of a pulmonary vein with an ablation device inserted therein.

FIG. 2 is a cross-sectional view of the apparatus in FIG. 1.

FIG. 3 is a close-up view of the ablation apparatus according to one embodiment of the invention, positioned inside a pulmonary vein.

FIG. 4 is a cross-sectional view of the ablation apparatus according to one embodiment of the invention.

FIG. 5 is a graph of the signals received from electrodes of an apparatus according to one embodiment of the invention.

FIG. 6 is a diagrammatic view of the ablation apparatus according to one embodiment of the invention.

FIG. 7 is a diagrammatic view of a portion of the ablation apparatus according to one embodiment of the invention.

### DETAILED DESCRIPTION

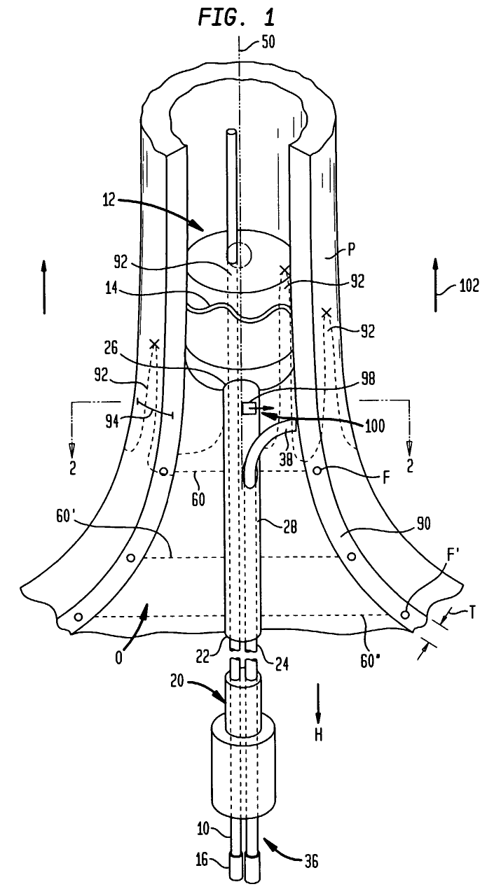

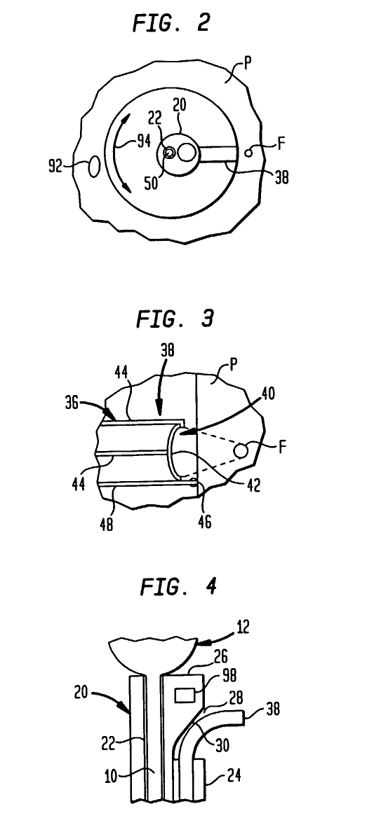

Apparatus according to one embodiment of the invention includes an elongated guide element 10, which may be a conventional, small diameter guide wire or catheter. Guide element 10 has an expansible anchor 12 mounted adjacent a distal end of the guide element. Anchor 12 may be a balloon or other structure movable between a collapsed condition in which the anchor closely surrounds the guide element 10 and the expanded condition illustrated in FIG. 1, in which the guide element projects radially from the guide element. The anchor has an electrode 14 extending around its circumference. The electrode is connected to one or more leads (not shown) extending in or on the guide element to the proximal end 16 of the guide element. The apparatus further includes a carrier catheter 20 having a guide lumen 22 and a treatment catheter lumen 24 extending in the lengthwise or proximal to distal direction of the carrier catheter. The guide lumen 22 extends to an opening at the distal end 26 of the carrier catheter. The treatment catheter lumen 24 terminates slightly short of the distal end. A port 28 in the side or circumferential wall of the carrier catheter communicates with the treatment catheter lumen at the distal end of this lumen. As best seen in FIG. 4, the carrier catheter desirably has a sloping wall surface 30 at the distal end of lumen 24. This wall surface slopes outwardly, towards port 28 in the distal direction.

The apparatus further includes a treatment catheter 36 having a distal end 38 and a small ultrasonic transducer 40 mounted at such distal end. The ultrasonic transducer is a piezoelectric element having a concave emitting surface 42 facing in the distal direction of the treatment catheter, i.e., to the right as seen in FIG. 3. The ultrasonic emitter is connected to leads 44 (FIG. 3) extending on or in the treatment catheter. These leads extend to the proximal end of the treatment catheter.

An electrode 46 is also mounted at the distal end 38 of the treatment catheter and connected to a further lead 48 extending on or in the treatment catheter.

In a method according to one embodiment of the invention, guide element 10 and anchor 12 are positioned as illustrated in FIG. 1, with the guide element extending through the subject circulatory system and through the left atrium of the subject's heart H into a pulmonary vein P through ostium or opening O of the vein. Anchor 12 is expanded to engage the wall of the pulmonary vein. Desirably, anchor 12 has a substantially cylindrical shape, and tends to bring the region of the pulmonary vein adjacent the anchor to a generally cylindrical cross sectional shape as well. In this condition, the axis 50 of the guide element 10, adjacent the distal end of the guide element lies substantially in the lengthwise direction of the pulmonary vein. Desirably, axis 50 is positioned by balloon 12 at or near the center of the vein. In the expanded condition of the anchor, the electrode 14 on the balloon is engaged with the wall of the vein.

Before or after expansion of the anchor, carrier catheter 20 is advanced to the position illustrated in FIG. 1. In this position, the guide element 10 extends through the guide lumen 22 of the carrier catheter, and the distal end 26 of the carrier catheter is disposed adjacent the anchor or balloon 12. For example, the distal end of the carrier catheter may abut the anchor so that the anchor prevents movement of the carrier catheter in the distal direction along the guide element.

Treatment catheter 36 is advanced within the treatment lumen 24 of the carrier catheter. When the treatment catheter reaches the distal end of lumen 24, it encounters sloping surface 30 and bends outwardly, through port 28 so that the distal end 38 of the treatment catheter protrudes from the carrier catheter. In this operative condition, the distal end of the treatment catheter is remote from axis 50. As the treatment catheter is advanced, electrical signals appearing at electrode 46 may be monitored. When the electrode contacts the wall of the pulmonary vein, the characteristics of such signal will change. In particular, the amplitude of naturally occurring electrical signals detected by the electrode will increase. Thus, by detecting this increase using a conventional monitoring device (not shown) connecting through lead 48 to the electrode, the physician can determine when the distal end 38 of the treatment catheter has been engaged with the wall of the pulmonary vein. To enhance this detection, a low voltage marker signal may be applied on electrode 14 at a frequency distinct from the frequencies of naturally occurring electrical signals. The electronic apparatus used to detect the voltage appearing at electrode 46 may be arranged to provide enhanced sensitivity to the marker signal and to suppress response to naturally occurring signals. For example, the detection apparatus may incorporate a frequency selective filter having a relatively narrow pass band centered at the marker frequency, or a synchronous detector locked to the marker signal.

Once the distal end of the treatment catheter has been engaged with the wall of the pulmonary vein, a drive signal is applied through leads 44 to ultrasonic transducer 40, causing it to emit ultrasonic waves. The ultrasonic waves converge with one another and mutually reinforce one another within a focal spot F. The position of the focal spot relative to the emitting surface depends, inter alia, on the curvature of the emitting surface. Desirably, this curvature is selected so that the focal spot lies within the wall of the pulmonary vein, beneath the surface of the vein wall lining. The applied ultrasonic energy heats and ablates the tissue of the vein wall. While the ultrasonic energy is being applied, carrier catheter 20 is rotated as, for example, by the physician manually turning the proximal end of the carrier catheter. The distal end of the carrier catheter rotates about axis 50. Stated another way, the guide element acts as a shaft received within the guide lumen 22, and the carrier catheter rotates about the shaft. The guide element substantially constrains the carrier catheter against movement transverse to axis 50. As the carrier catheter rotates, the distal end 38 of the treatment catheter sweeps along an arcuate path 60 substantially concentric with axis 50 on the vein wall. The focal spot F traces a similar path within the vein wall. Thus, the ultrasonic energy ablates tissue within an arcuate zone. A complete, loop like path 60 around the entire pulmonary vein may be ablated by turning the distal end of the carrier catheter through a complete, 360° rotation. Engagement of the treatment catheter distal end with the vein wall may be monitored during this procedure by monitoring the voltage on electrode 46, and the treatment catheter may be moved relative to the carrier catheter to maintain such engagement. Resilience of the treatment catheter, carrier catheter, the guide element and anchor also help to maintain engagement even if the vein wall is not perfectly circular.

This procedure provides ablation of a complete circumferential loop using a small, localized ultrasonic treatment element. Moreover, such a loop can be formed without depending entirely upon the physician's technique in maneuvering the catheter. That is, the distal end of the catheter is guided in its motion around the circumference of the pulmonary vein.

In the method discussed above, the path 60 of the focal spot extends around the wall of the pulmonary vein itself. However, as is well known in the treatment of atrial fibrillation, a conduction block can be formed at any location proximal to the focus X of the arrhythmia, which is typically located at a point along the pulmonary vein. For example, an effective conduction block can be formed in precisely the same manner along an alternate path 60′ in the wall of the ostium, provided that the ablation capabilities of the treatment catheter allow effective ablation through the thickness T of the myocardial tissue in the ostium. Likewise, the same techniques can be used to form a conduction block in the wall of the heart along a path 60″. The treatment catheter 38 would extend further from the axis 50 to inscribe a larger circular path. Also, anchor 12 would be positioned proximally from the location shown as, for example, within the ostium of the pulmonary vein rather than deep within the pulmonary vein itself.

In the techniques discussed above, the conduction block is formed as a complete, closed loop extending 360° around axis 50. However, as further described in the aforementioned Ser. No. 60/265,480 application, now U.S. patent application Ser. No. 10/062,693, there is a boundary or border 90 between myocardial tissue in the heart wall H and vein wall tissue of the pulmonary vein P. In patients suffering from atrial fibrillation, abnormal fibers 92 of myocardial tissue extend distally from this border along the pulmonary vein P. The abnormal electrical impulses associated with atrial fibrillation are transmitted from the focus X of the arrhythmia along these abnormal fibers 92. Thus, if ablation is performed at a location between the border 90 and the focus X of the arrhythmia, transmission of the abnormal electrical impulses can be halted by ablating the abnormal myocardial fibers 92. In this instance, it is only necessary to ablate along a path encompassing the abnormal myocardial fibers; it is not necessary to ablate along a complete, closed loop around the entire circumference of the pulmonary vein. For example, ablation along a path 94 distal to border 90 and encompassing fibers 92 is sufficient to inhibit transmission of the abnormal electrical impulses, assuming that these are the only abnormal myocardial fibers in the particular pulmonary vein.

Ablation over a limited path is advantageous for several reasons. The degree of damage to normal tissue will be less than with ablation along a complete loop. This tends to reduce the possibility of thrombus formation and stenosis of the pulmonary vein. Also, the procedure can be performed in a shorter time.

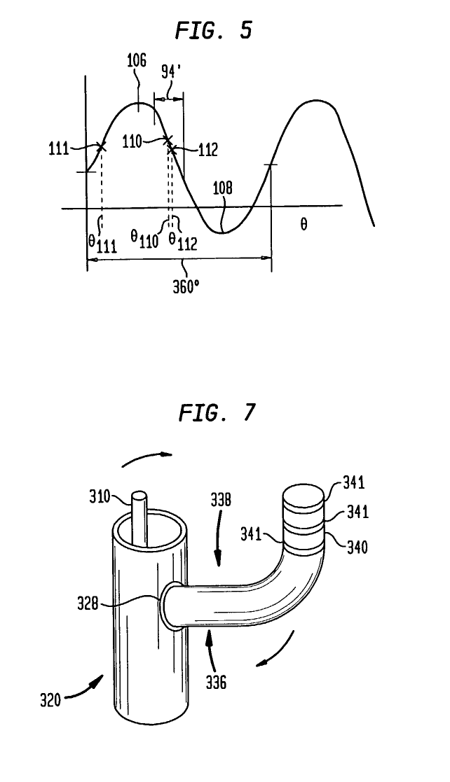

In a further embodiment of the present invention, a sensor 98 is provided on carrier catheter 20 adjacent the distal end thereof. Sensor 98 is arrange to provide a signal which depends upon the alignment between a sensing direction, indicated as vector 100 on the sensor and the direction of a magnetic or electromagnetic field 102 prevailing in the vicinity of the sensor. For example, sensor 102 may be a hall effect sensor, magneto resistive sensor or the like having an output voltage which varies with the component of a magnetic field in the sensing direction 100. In a method using this sensor, the anchor, guide element, carrier catheter and treatment catheter are positioned as discussed above so that the distal end 38 of the treatment catheter is disposed distal to the border 90 between myocardial tissue and vein wall tissue. In a fiber-locating step, carrier catheter 20 is rotated about axis 50 so it can sweep the distal end of the treatment catheter along path 60. However, in this stage of operation, the ultrasonic element 40 is not actuated to ablate the tissue. Rather, the ultrasonic element is used as an echo detection device. Thus, the ultrasonic element is actuated intermittently with a low power echo-sounding drive signal. During intervals between such actuations, the transducer serves to convert ultrasonic waves reflected by the tissue in front of the transducer into electrical signals representing the echoes from the tissue. Because the ultrasonic properties of myocardial fibers differ from the ultrasonic properties of vein wall tissue, the electrical signals generated by the transducer when the transducer is aligned with a fiber 92 will differ from those generated when the transducer is not aligned with a fiber. As the carrier catheter and sensor 98 rotate during this step, the voltage from the sensor will vary with the angular position θ of the carrier catheter and hence with the angular position of the treatment catheter distal end 38. For example, as indicated in FIG. 5, the voltage will be at a maximum at point 106 where the sensing direction 100 (FIG. 1) is most nearly co-directional with the field direction 102 and at a minimum at another value of θ at 108, where the sensing direction 100 is most nearly opposite (counter-directional) to the field direction 102. This variation will occur for any field direction 102, provided that the field direction is not exactly parallel to axis 50. Thus, the angular position θ of the carrier catheter can be monitored by monitoring the signal voltage from sensor 98. Although the angular position is not a unique function of signal voltage, the angular position can be determined from the signal voltage. For example, a particular value f signal voltage occurs at two points: θ₁₁₁ and θ₁₁₂ within 360° of rotation. However, at point 111 the signal voltage increases with rotation in a particular direction, whereas at point 110 the signal voltage decreases with rotation in this direction. Only point 110 exhibits the combination of the same voltage and this trend or slope in the voltage versus rotation curve.

The results of the ultrasonic monitoring step plotted against rotational position. Those rotational positions associated with ultrasonic results indicating the presence of myocardial fibers are identified. For example, assume that the distal end 38 of the treatment catheter is aligned with myocardial fibers 92 at rotational positions θ₁₁₀ and θ₁₁₂.

Once the rotational positions associated with myocardial fibers have been identified, the carrier catheter and treatment catheter are rotated through a range of rotational positions encompassing the rotational positions associated with the myocardial fibers as, for example, the range 94′ (FIG. 5) encompassing rotational positions θ₁₁₀ and θ₁₁₂, so as to sweep the distal end of the treatment catheter over the path 94 encompassing the myocardial fibers 92 (FIG. 1). During this step, the transducer 40 is actuated to ablate the vein wall tissue in the manner discussed above and thus ablate the abnormal myocardial fiber 92.

In a variant of the procedure discussed above, the fiber locating step can be performed using electrode 46 rather than transducer 40 as the sensing element. Thus, a marker signal as discussed above is applied through electrode 14. Because myocardial fibers 92 will conduct electrical signals differently than the normal tissue of the vein wall, the marker signal will appear at greater amplitudes when the electrode 46 (FIG. 3) on the distal end of the treatment catheter is aligned with a myocardial fiber.

In a further variant of the procedures discussed above, the sensor can be carried on treatment catheter 38, rather than on the carrier catheter. Indeed, treatment catheter 38 may be a commercially available electrophysiological ablation catheter equipped with a position sensor. In yet another variant, the fiber locating step can be performed using a locating catheter (not shown) inserted through treatment lumen 24. The locating catheter may carry any type of sensor capable of identifying the presence of myocardial fibers, including the ultrasonic and electrode sensors discussed above. After the locating step, the locating catheter is withdrawn and the treatment catheter is inserted into the treatment lumen of the carrier catheter as discussed above.

There is a repeatable association between the position of the treatment catheter and the rotational position of the carrier catheter distal end. Because the rotational position of the carrier catheter distal end is monitored either directly using a sensor on the carrier catheter itself or indirectly using a sensor on the treatment catheter, the procedure does not depend upon accurate transmission of rotation between the proximal end of the carrier catheter and the distal end. However, translational movement of the carrier catheter relative to the guide element typically can be transmitted from the proximal ends of these devices to their distal ends with good accuracy and repeatability.

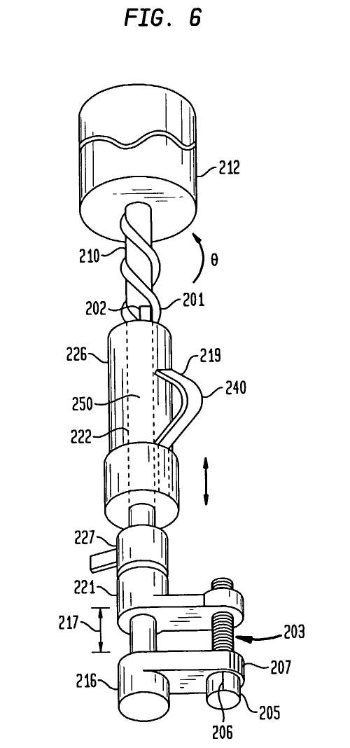

As shown in FIG. 6, in an apparatus according to a further embodiment of the invention, the distal end 226 of carrier catheter 220 and the adjacent portion of guide element 210 are interconnected by a translation to rotation conversion mechanism including a helical cam 201 on the guide element and a mating follower surface 202 on the carrier catheter. The opposite arrangement (helical surface on carrier catheter with follower on guide element) can also be used. Any other mechanical elements are capable of converting translation of the distal end 226 relative to the guide element 210 into a rotation of the carrier catheter distal end relative to the guide element can be used. Thus, the distal end 226 of the carrier catheter can be brought to a repeatable rotational position relative to the anchor 212 and relative to the adjacent tissues (not shown) by controlling the position of the proximal end 221 of the carrier catheter relative to the proximal end 216 of the guide element.

A conventional position controlling mechanism such as a screw mechanism 203 interconnects the proximal ends 221 and 216 so that the distance 217 between these ends may be varied as desired in a controlled manner. A conventional indicating device such as a knob 205 associated with screw mechanism 203 and a scale 207 associated with a pointer 206 on the knob is provided for indicating the distance 217. Each value of distance 217 corresponds to a particular value of the angular position θ of the distal end 226 relative to anchor 212 and guide element 210. Thus, there is no need to detect the angular position relative to a field as discussed above. The myocardial fiber locating step can be performed as discussed above, and the linear positions on scale 207 corresponding to the locations of myocardial fibers can be recorded. In the ablation step, the carrier catheter is moved relative to the guide element through a range of linear positions sufficient to encompass the linear positions associated with the myocardial fibers, thus sweeping the ablation element over a range of angular positions which encompass the myocardial fibers during the ablation step. The same apparatus can be used to perform a full-loop, 360° ablation as discussed above, without the need for a locating step, by moving the carrier catheter relative to the guide catheter through a range of linear positions corresponding to a full 360° rotation.

Any other form of mechanical positioning device may be substituted for screw mechanism 203. Also, the dial and scale 205 and 207 may be replaced by any other conventional device for monitoring the relative positions of the two elements as, for example, a mechanical dial indicator or an optical or electronic position measuring device.

In the apparatus of FIG. 6, the ablation element 240 is not carried on a separate treatment catheter. Rather, the ablation element is mounted on a deformable element such as a strip 219. In the extended position depicted in FIG. 6, the leaf-life element projects in the radial direction from the carrier catheter so that the ablation element 240 is removed from the axis 250 of the guide board 222 in the carrier catheter and hence remote from the axis of rotation of the carrier catheter around guide element 210. In the collapsed condition (not shown) leaf element 219 lies against the side wall of carrier catheter 220 to facilitate threading. The resilience of leaf element 219 normally biases to the collapsed condition. A sleeve or other axially moveable element 227 carried on the carrier catheter can be actuated from the proximal end of the carrier catheter to move the leaf element to the extended condition. Any other type of radially expansible structure as, for example, a balloon, can be used instead of the leaf element.

Apparatus according to a further embodiment of the invention (FIG. 7) incorporates a carrier catheter 320 and guide element 310 similar to the corresponding elements discussed above with reference to FIG. 1. However, the treatment catheter 336 has distal end 338 adapted to form a generally J-shaped configuration when the treatment catheter is extended through the port 328 on the carrier catheter. The ablation element 340 includes a series of sub-elements 341 such as electrodes for RF application or ultrasonic transducers encircling the distal end of the treatment catheter. The side wall of the treatment catheter distal end in the vicinity of ablation element 340 is engaged with the wall of the pulmonary vein, ostium or heart when the treatment catheter is in the extended position illustrated.

In yet another variant, the sensor 98 discussed above with reference to FIG. 1 can be replaced by a rotary position encoder having one element linked to the distal end 26 of carrier catheter 20 and another element linked to anchor 12. Such a rotary position encoder is arranged to provide a signal representing the angular position of the carrier catheter with respect to the anchor. Because the anchor remains in a fixed position relative to the pulmonary vein, this angular position can be used in the same manner as the angular position of the carrier catheter with respect to a field.

The particular ablation elements discussed above are merely exemplary. For example, the treatment catheter may include an optical fiber for transmitting intense light from a source such as a laser from the proximal end of the catheter so that the light ablates the tissue. Alternatively, the treatment catheter may be a tubular catheter adapted to conduct a chemical ablation agent to an outlet at the distal end. In yet another alternative, the treatment catheter may carry a blade or other mechanical device for mechanically ablating (cutting) the tissues to a controlled depth.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating cardiac arrhythmias, comprising:
locating mycocardial fibers within a pulmonary vein;
ablating said myocardial fibers within said pulmonary vein;
determining whether electrical signals can be propagated through said myocardial fibers such that arythmogenic signals can be transmitted; and
repeating said ablating step if said electrical signals can be propagated through said myocardial fibers such that said arythmogenic signals can be transmitted.

2. A method as claimed in claim 1, further comprising the steps of:
applying a signal to said myocardial fibers; and
determining the magnitude of such signal transmitted trough said myocardial fibers.

3. A method as claimed in claim 2, further comprising the step of determining the magnitude of such signals around the circumference of said pulmonary vein.

4. A method as claimed in claim 2, further comprising the step of determining whether the ablation device is in contact with the wall of the pulmonary vein.

5. An ablation apparatus, comprising:
(a) a carrier catheter having a proximal and a distal end, a longitudinal bore from said proximal to said distal end, an axis, and an aperture communicating with said longitudinal bore, said aperture being between said proximal and said distal end;
(b) a treatment catheter having a distal portion, said distal portion disposed slidably within said longitudinal bore, wherein said distal portion is positionable such that at least a portion of said distal portion of said treatment catheter can pass through said aperture and project away from said axis; and
(c) an ablation device on said distal portion of said treatment catheter, whereby rotating said carrier catheter about said axis causes said ablation device to traverse a circular path.

6. An ablation apparatus as claimed in claim 5, further comprising a device to determine the angular rotation of said carrier catheter about said axis.

7. An ablation apparatus comprising:
(a) at least one catheter having a proximal end and a distal end, said at least one catheter including a distal portion adjacent said distal end, said distal portion defining an axis and radial directions transverse to said axis;
(b) an ablation device carried on said at least one catheter, said ablation device being movable between an inoperative position and an operative position in which said ablation device is adjacent said distal portion of said catheter and is remote from said axis, said ablation device moving with a component of motion in a radially outward direction in movement from said inoperative position to said operative position; and
(c) a rotation drive mechanism linked to said ablation device and operative to swing said ablation device about said axis while said ablation device is in said operative position.

8. Apparatus as claimed in claim 7 wherein said at least one catheter includes a carrier catheter and a treatment catheter, said ablation device being mounted on said treatment catheter.

9. Apparatus as claimed in claim 8 wherein said rotation drive mechanism is connected between said carrier catheter and said treatment catheter, and wherein said rotation drive mechanism is operative to rotate said treatment catheter relative to said carrier catheter.

10. An apparatus for positioning a catheter, comprising:
a carrier catheter having a proximal end, a distal end, a longitudinal bore from said proximal to said distal end, and an engaging portion between said proximal end and said distal end; and
a treatment catheter having a distal portion and a mating portion, said distal portion disposed slidably within said longitudinal bore, wherein said mating portion engages with said engaging portion of said carrier catheter such that moving said treatment catheter slidably within said carrier catheter rotates said treatment catheter.

11. An apparatus for positioning a catheter, comprising:
a carrier catheter having a proximal end, a distal end, and an engaging portion between said proximal end and said distal end; and
a treatment catheter having a distal portion and a mating portion, said distal portion disposed slidably on said carrier catheter, wherein said mating portion engages with said engaging portion of said carrier catheter such that moving said treatment catheter slidably on said carrier catheter between said proximal end and said distal end rotates said treatment catheter.

12. An apparatus for positioning a catheter, compromising:
a carrier catheter having a proximal portion and a distal portion, said proximal and distal portions defining an axis;
a second catheter having a distal portion, and an distal end, said distal portion of said second catheter disposed slidably relative to said carrier catheter such that sliding said second catheter relative to said carrier catheter results in movement of the distal end of said second catheter transverse to said axis of said carrier catheter.

13. An apparatus as claimed in claim 12, wherein said transverse movement is radial to said axis.
